(19) United States
(12) Patent Application Publication
(10) Pub. No.: US 2025/0375572 A1
(43) Pub. Date: Dec. 11, 2025

(54) METHODS AND APPARATUS FOR MONITORING INTRAVENOUS CATHETER INFILTRATION

(71) Applicant: Board of Regents, The University of Texas System, Austin, TX (US)

(72) Inventors: Daniel Dearmond, San Antonio, TX (US); John Calhoon, San Antonio, TX (US)

(21) Appl. No.: 19/212,587

(22) Filed: May 19, 2025

Related U.S. Application Data
(60) Provisional application No. 63/649,393, filed on May 19, 2024.

(51) Int. Cl. A61M 5/168

(52) U.S. Cl. CPC A61M 5/16836; A61M 2205/18; A61M 2205/3331; A61M 2205/502; A61M 2205/581; A61M 2205/582; A61M 2205/583; A61M 2210/12

(57) ABSTRACT

Embodiments are directed to an automated system that provides statistical confirmation of the non-infiltrated state of a functioning PIV and a clear signal associated with an infiltrated PIV identifying conditions associated with infiltration/extravasation before rather than after the onset of a noxious event such as tissue edema or damage.

### RELATED APPLICATIONS

This application claims priority to U.S. Provisional Patent Application No. 63/649,393 filed May 19, 2025, which is incorporated herein by reference in its entirety.

### STATEMENT REGARDING FEDERALLY FUNDED RESEARCH

None.

### FIELD

Embodiments generally relate to medicine and the intravenous delivery of fluids to a subject. More particularly the embodiments relate to systems and methods of monitoring intravenous catheter/cannula-associated venous occlusion, infiltration or extravasation using a smart phone or a smart watch-type applications with novel graphical interfaces.

### BACKGROUND

Worldwide an estimated two billion peripheral intravenous catheters (PIVs) are placed each year making PIV placement one of the most common medical procedures performed and PIVs one of the most common medical devices in current medical practice (Hirata et al., ACS Sensors 2023 8(3), 1017-32). A common complication of PIVs is the phenomenon referred to as extravasation or infiltration affecting as many as 40% of all PIVs (Cheng et al., IEEE 66th Electronic Components and Technology Conference (ECTC), 2016:1632-37). Though the pathophysiology of PIV infiltration or extravasation is not completely described and is likely multifactorial, the common final pathway is that the PIV is not seated in a patent vein, which denies fluids and/or medications introduced into the PIV a low-resistance pathway to the central venous system and heart and diverts them instead into the soft tissues in the direct surroundings of the PIV puncture site. In some circumstances the catheter itself, as a relatively rigid foreign body in a thin-walled peripheral vein, could rupture the vein directly such that the tip of the PIV migrates outside the lumen of the vein. Intravenous fluids introduced through the PIV would then directly enter the surrounding soft tissues causing edema. Catheter trauma involving damage to the tissues of the vein and/or immediately surrounding sub-cutaneous tissues may also lead to an inflammatory response in the vicinity of the vein causing local edema with extrinsic compression and blockage of the vein and re-routing of IV fluids/medications into the soft tissue around the puncture site of the PIV through the damaged vein wall. Alternatively, the presence of the foreign material of the PIV in the peripheral vein may lead to conditions favoring peripheral vein thrombosis thereby cutting off communication of the PIV with the more central venous system. Regardless of the mechanism, with the PIV not seated in a patent vein, fluids and/or medications delivered by an intravenous (IV) fluid pump into the PIV and intended for delivery via the PIV to the greater blood circulation are diverted instead into the soft tissues surrounding the site of PIV placement resulting in pain/discomfort to the patient, skin redness or edema/swelling, and in extreme cases skin and/or soft tissue necrosis due to tissue damage from caustic medications (Hirata et al., ACS Sensors 2023 8(3), 1017-32). Neonates, children, and the elderly are particularly susceptible to the complications of PIV infiltration/extravasation (Gault, British Journal of Plastic Surgery, Vol 46 (2), 1993, 91-96).

The vigilance and experience of nurses or other clinicians to identify early infiltration/extravasation by visual assessment and/or palpation of local skin conditions in the vicinity of the PIV remains the primary agency of preventing complications (Hirata et al., ACS Sensors 2023 8(3), 1017-32). However, this system relies on labor-intensive, subjective methods and produces inconsistent outcomes. Electronic IV fluid pumps in common usage for IV fluid and medication delivery have built-in high pressure warning systems that are intended to trigger in the setting of IV infiltration/extravasation but these are only partially effective. There is wide consensus for the need of an improved means of monitoring for PIV infiltration/extravasation and a proliferation of technologies have been proposed to address this condition (Hirata et al., ACS Sensors 2023 8(3), 1017-32).

The inventors previously described a system for introducing pulses into a PIV/peripheral vein system by applying a signaling actuator to the intravenous tubing connected to the PIV outside the body (4). This allowed for the introduction of a pulse of very-low force (1-3 mm H₂O) into the PIV/peripheral vein. Given that the PIV/peripheral vein in normal operation has very low signal-to-noise in terms of fluid dynamics, the pulse signal, despite being very low in force, was readily detectable by ultrasound or other modalities. The pulse signal was abrogated upon occlusion of the vein indicating that propagation of the pulse signal required an intact fluid column, i.e., a continuous hydraulic system encompassing the intravenous tubing outside the body, the PIV, and the peripheral vein (U.S. Pat. No. 10,842,931). The '931 patent describes methods and apparatus for generating and detecting a pulse in a fluid column in IV tubing connected to a PIV in a vein, the downstream vein will always be patent, therefore, the presence of a pulse in the IV tubing can be taken as a surrogate for non-infiltrated status of the vein; if a pulse cannot be generated and detected in the IV tubing fluid column, in the absence of other obstruction of the IV tubing or malfunction of the measuring equipment, the vein downstream of the IV tubing can be concluded to be non-patent or infiltrated. These findings have been reinforced over numerous in vivo and in vitro experiments. No means of automating the process of pulse introduction or interpretation for statistical analysis was described in '931 patent. Overall, IV infiltration poses significant risks and challenges, highlighting the importance of proper technique, vigilant monitoring, and prompt intervention to minimize complications and ensure patient safety during intravenous therapy.

There remains a need for additional methods and apparatus for monitoring and detecting the presence of infiltration during medical interventions.

### SUMMARY

A solution to the problems associated with IV infiltration is provided by an automated system that provides statistical confirmation of the non-infiltrated state of a functioning PIV and a clear signal associated with an infiltrated PIV to improve clinician and patient confidence that a PIV was in a functioning state prior to using it to deliver fluids or medications. Aspects of the invention reduce nurse/clinician workload by eliminating the need for routine removal and replacement of functioning PIVs as necessitated per current hospital protocols. Other aspects of the invention eliminate the time-consuming and potentially subjective and inconsistent nature of nurse/clinician clinical diagnosis of infiltrated PIVs. Still other aspects of the invention reduce potentially severe patient complications associated with PIV infiltration/extravasation. Optimally such a system would identify conditions associated with infiltration/extravasation antecedent to rather than subsequent to the onset of a noxious event such as tissue edema or damage.

Herein, the inventors describe a system building on the original pulse-introduction system that allows for automation of the introduction, detection, and interpretation of pulse signals in a PIV/peripheral vein as a means of identifying PIVs that have infiltrated so as to avoid the complications associated with PIV extravasation.

Certain aspects of the problems associated with intravenous infiltration have been solved by engineering a device which attaches to an intravenous catheter that transmits a pulse signal to the intravenous fluid column and developing a method that interprets a signal induced by the transmitted pulse. The device and/or method confirms non-infiltration and proper in-vein placement of the intravenous catheter in a fully automated manner which does not rely on health provider interpretation.

Certain embodiments are directed to graphical interface for application devices including a smart watch or smart phone based on frequency spectra of induced pulse patterns allowing for rapid, continuous statistical confirmation of catheter patency to improve intravenous catheter infiltration monitoring.

In an embodiment, an in-line impulse generator is incorporated into the IV tubing outside of a patient's body connected to a PIV catheter for the purpose of introducing pulses into the IV tubing, the PIV, and the peripheral vein, which otherwise do not contain pulsatility. The introduced pulse would be detected as fluid movement in the IV fluid line upstream from the peripheral vein by a velocimetry sensor and converted to a data signal which would be analyzed by a programmable logic circuit (PLC). Algorithmic analysis of the pulse signal by the PLC would determine whether the received velocimetry data signal was congruent with the expected, introduced pulse pattern within a statistical confidence interval. This would result in a clear, graphical and alphanumeric representation of the statistical analysis on the screen of a smart watch or smart phone-type device which would be readily interpretable by patient and care-team members as confirmation of, or failure to confirm, a non-infiltrated PIV safe for use.

In certain embodiments the system can be configured to monitor and assess a number of patient data and/or hospital data, identifiable or non-identifiable, and may include biometric parameters of a patient or staff treating the patient. Biometrics refers to the measurement and analysis of unique physical or behavioral characteristics used to identify or authenticate individuals. These characteristics are distinctive and measurable. Types of biometrics include physical biometrics and behavioral biometrics. Physical biometrics are based on biological traits. Behavioral biometrics are based on patterns in behavior. A biometric system captures an individual's biometric data (e.g., a fingerprint scan) and stores it as a template in a database. Authentication (1:1 matching) compares the captured biometric data against a stored template to verify identity (e.g., unlocking a phone with a fingerprint). Identification searches a database to find a match for the biometric data. Algorithms analyze the biometric data, converting it into a digital format for comparison while accounting for minor variations (e.g., slight changes in lighting for facial recognition). Certain aspects are used in conjunction with wearable biometrics in devices like smartwatches for continuous authentication. Health data that could be collected for a patient includes any information related to their physical, mental, or behavioral health. This data can be gathered through various sources like medical records, wearable devices, patient self-reports, or clinical observations. Health data can include one or more of demographic information such as age, sex, gender, race, ethnicity; contact details (address, phone, email); marital status, occupation, education level; vital signs (blood pressure, heart rate, respiratory rate, body temperature, oxygen saturation (SpO₂); medical history (past and current medical conditions, surgical history, allergies, immunization records, family medical history (genetic or hereditary conditions); medications and treatments; etc. This data can be collected via electronic health records (EHRs), patient portals, mobile apps, wearable devices, surveys, or direct clinical interactions. The specific data collected depends on the patient's condition, healthcare provider's goals, and the context of care (e.g., routine checkup vs. chronic disease management).

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps) but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1A-1B depicts an overview of the induced-pulse velocimetry signaling (IPVS) system.

FIG. 2A-2B depicts a miniaturized, wearable version of the IPVS unit of FIG. 1A-1B with the internal components shown in FIG. 2A and the outer case with display screen shown in FIG. 2B.

FIG. 3 depicts the self-contained, wearable version of the IPVS unit from FIG. 2 in place along the arm with connections to a PIV and an IV fluid tubing line.

FIG. 4A-4B depicts a magnified view of the velocimetry signal detection sub-unit of the IPVS system using a laser/laser-sensor mechanism for obtaining pulse velocimetry values.

FIG. 5A-5B shows inter-pulse interval values (in seconds) obtained experimentally from the IPVS using the laser/laser sensor mechanism for a one-second inter-pulse interval (FIG. 5A) and a two-second inter-pulse interval (FIG. 5B).

FIG. 6 depicts a test performance measure table for IPVS with respect to true disease status of PIV/vein infiltration present versus absent.

FIG. 7A-7C depicts data tables for the expected inter-pulse interval sequence (i.e., the sequence delivered by the pulse actuator) (FIG. 7A), the observed inter-pulse interval sequence (i.e. the sequence actually measured by the signal detection system) (FIG. 7B), and a merged data table in which the expected and observed values are enmeshed (7C).

FIG. 8A-8B depicts a fast Fourier transform (FFT) of the 16-point inter-pulse interval data values shown in FIG. 7C with FIG. 8A showing the magnitude of the vectors of the FFT at the given frequencies and FIG. 8B showing the phase of the vectors of the FFT.

FIG. 9 depicts the FFT efference window with gray cells indicating where observed pulse values are inserted.

FIG. 10A-10B shows FFT plots generated by the programmable logic circuit in real time from experimental pulse data overlayed with a quadratic regression curve fitted to the magnitudes of the generated Fourier spectrum (including the R² value in the plot title) in a case of a signal match confirmation (A) and a case of failure to confirm a signal match (B).

FIG. 11 depicts the self-contained, wearable version of the IPVS unit with an electronic display to show vein infiltration status via the FFT derived from the generated pulse signal.

FIG. 12 depicts an experimental setup allowing for the testing of the impact of IV pulse on infection risk.

FIG. 13 depicts tubes with IV catheter flush fluid from an IV with a pulse and an IV without.

FIG. 14 depicts IV catheter fluid flush from IV with and without pulse on agar plates to show growth of bacteria.

FIG. 15 depicts the number of colonies grown from IV catheter fluid flush from IV with and without pulse. Pulsed IV shows large decrease in number of colonies.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to imply that the scope of the disclosure, including the claims, is limited to that embodiment.

Intravenous (IV) infiltration occurs when the infused fluid or medication leaks into the surrounding tissue instead of entering the vein as intended. This can lead to various problems and complications, including: (i) Tissue Damage: The infiltration of fluid or medication into the surrounding tissue can cause irritation, inflammation, and tissue damage. This can result in pain, swelling, and potential long-term complications such as scarring or tissue necrosis. (ii) Loss of Medication: When the medication infiltrates the tissue instead of entering the bloodstream, it may not achieve the desired therapeutic effect. This can result in inadequate treatment and potential complications related to the underlying condition. (iii) Delayed Treatment: Infiltration may lead to a delay in receiving essential medications or fluids, especially in critical care settings where timely administration is crucial for patient outcomes. (iv) Infection Risk: The presence of foreign substances in the tissue can increase the risk of infection. Bacteria introduced during infiltration can cause localized infections at the site, which may require antibiotic treatment and can lead to systemic complications if left untreated. (v) Compromised IV Access: Infiltration can damage the integrity of the vein, making it difficult to establish or maintain IV access in the affected limb. This can be particularly problematic in patients with limited venous access, such as those with chronic illnesses or compromised vascular systems. (vi) Fluid Overload: In some cases, significant infiltration of fluids can lead to fluid overload, especially if large volumes of intravenous fluids are administered rapidly. This can result in complications such as pulmonary edema, congestive heart failure, or electrolyte imbalances. (vii) Pain and Discomfort: Patients may experience pain, discomfort, or a burning sensation at the site of infiltration. This can contribute to patient distress and may require additional interventions to manage symptoms. (viii) Complications in Special Populations: Certain patient populations, such as infants, elderly individuals, and those with fragile or compromised skin, may be at increased risk of complications from IV infiltration due to their unique physiological characteristics.

System of intravenous fluid/medication delivery that employs rapid automated algorithmic analysis of exogenously introduced flow amplitudes, frequencies or pulses to identify intravenous catheter infiltration, occlusion, or out-of-vein placement. Novel device for measuring fluid velocimetry in a vein (e.g., laser break-beam). Assessment of IV patency/lack of infiltration using a novel machine-learning algorithm: efference spectral framing (ESF) ESF allows for completely machine-directed IV infiltration monitoring without the need for healthcare provider interpretation. Additionally, the pulsing can decrease the risk of infection in the IV catheter.

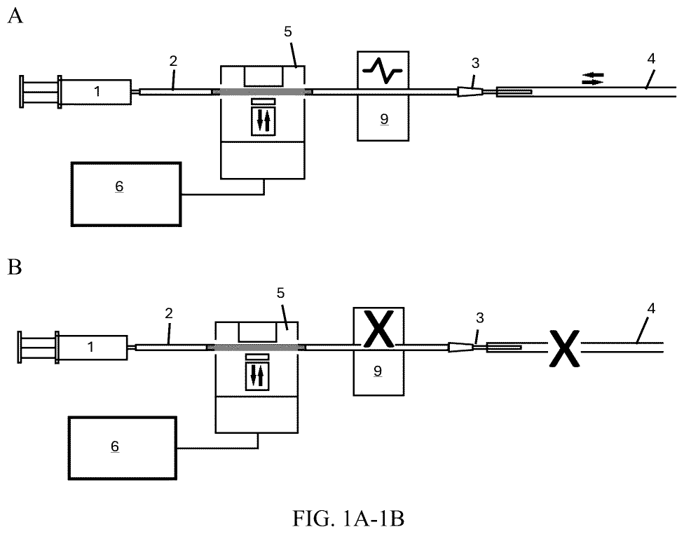

FIG. 1 shows a conceptual overview of the induced-pulse velocimetry signaling (IPVS) system which has been previously described. In FIG. 1A, syringe 1 containing electrolyte serves as the fluid reservoir for the system and is connected to IV tubing 2 which in turn is connected to a PIV 3 which is inserted into the vein of a patient 4; all of these structures are contiguously fluid filled with electrolyte fluid and/or medications or in some embodiments colloid fluids or blood products. In some embodiments the electrolyte reservoir would be a bag of electrolyte fluid connected to an IV pump to advance fluids and/or medications into the vein of the patient through the PIV. In the IPVS system a pulse generator 5 acts via an electromagnetic motor 6 to apply back and forth displacement of the fluid in the IV tubing in a specific pulse pattern to generate a fluid pulse movement of the fluid inside the IV tubing 2 which is ultimately propagated as back and forth movement of the fluid column in the vein of the patient. In some embodiments the electromagnetic motor 6 of the pulse generator 5 could be replaced by a gear mechanism utilizing only analog information input to drive the pulse pattern. The forward movement phase of the fluid is due to the electromagnetic motor 6 inducing compression of the IV tubing 2, which is fabricated from a compressible plastic, latex, or silicone material; the return or backward movement phase of the fluid is due to elastic recoil of the IV tubing 2 as it regains its non-compressed state and pulls the IV fluid back through the IV tubing. The fluid velocimetry signal detection sub-unit 9 of the IPVS system detects the back and forth pulsatile movement of the fluid in the IV tubing 2 through ultrasound, optical-signaling, or some other velocimetry-detection mechanism and translates this into a digital or analog signal which can be transmitted by connecting wires to a programmable logic circuit (PLC) for analysis of the signal. The PLC can also connected to the pulse generator 5 by connecting wires and contains the digital programming for the specific pulse pattern delivered by the pulse generator. The PLC may be powered by a rechargeable or replaceable battery or an external power supply (not shown).

FIG. 1B depicts the findings of extensive in vitro and in vivo testing. It has been shown that if the PIV 3 (FIG. 1A) resides in an infiltrated or otherwise non-patent vein that cannot be made to undergo back and forth pulsatile movement, or if the PIV has migrated outside the lumen of the vein, or the PIV in some other way is in a condition in which the continuous fluid column extending from the electrolyte reservoir 1 (FIG. 1A) to the vein of the patient 4 (FIG. 1A) has been physically interrupted so as to abrogate the back and forth signal in the vein induced by the pulse generator 5 (FIG. 1A) then perforce the back and forth flow in the IV tubing upstream from the vein will also be abrogated leading to a loss of the velocimetry signal detected by the velocimetry signal detection sub-unit. Introduction of IV fluids or medications under these conditions (FIG. 1B) would be likely to result in infiltration/extravasation. This is the fundamental principle on which the IPVS system is predicated.

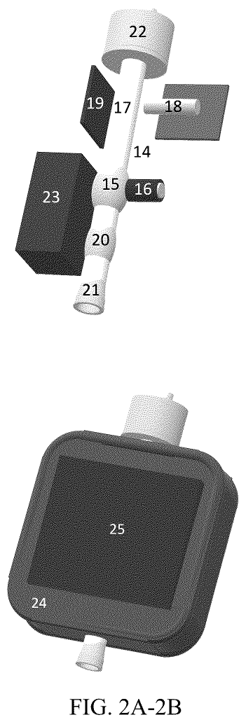

The components of the IPVS system described in FIG. 1A can be miniaturized to accommodate the format of a wearable medical device as has been previously described in detail (U.S. Pat. No. 11,666,700). FIG. 2A-2B depicts the internal components (FIG. 2A) and the outer case (FIG. 2B) of such a self-contained, wearable version of the IPVS unit. FIG. 2A depicts a segment of IV tubing 14 which is specifically engineered to accommodate all functionality of the IPVS (U.S. Pat. No. 11,666,700). This IV tubing segment 14 contains a compressible/recoilable section 15, which receives pulses from the electromagnetic motor of the pulse generator 16, and an optically engineered segment 17 which allows light to be passed through the tubing from a light source 18 to contact a light sensor 19. The tubing also has an optional dampener portion 20 and proximal 21 and distal 22 connection points for the IV fluid source and the IV catheter respectively as previously described (U.S. Pat. No. 11,666,700). A PLC 23 supplies power to all components, sends pulse signal data to the pulse generator 16 and receives velocimetry data from the light sensor 19 via connecting wires (not shown). In the embodiment shown these components are roughly 4 cm in maximum extent. In certain aspects the length along the long axis can be about 3, 4, 5, 6, 7, 8, 9, to 10 cm.

FIG. 2B shows the components from FIG. 2A contained in a protective case 24 on the surface of which is seated a digital display 25 similar to a smart watch or smart phone monitor screen which can be used to display graphical or alphanumeric data to the user.

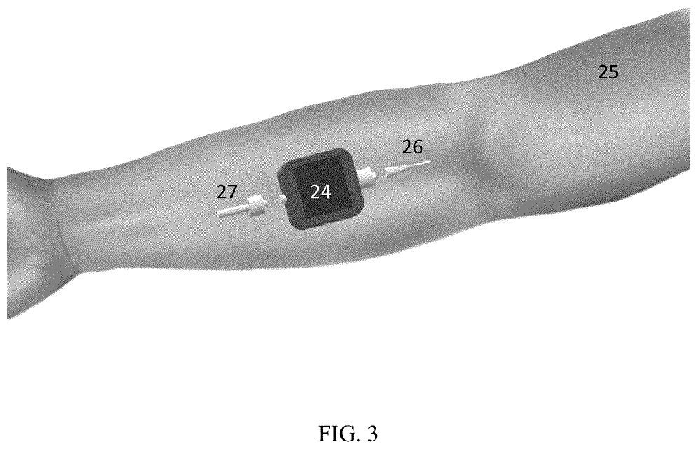

FIG. 3 shows the protective case of the wearable IPVS system 24 from FIG. 2B as it would appear on the forearm of a patient with a PIV. The arm of the patient 25 in this case has a PIV 26 placed in the antecubital fossa which connects to the wearable IPVS system 24. The wearable IPVS 24 is also connected upstream to an additional IV connector 27 which in turn would be connected to a source of fluids or medications (not shown) or alternatively, for a PIV that was not actively being used to deliver fluids or medications this IV connecting end 27 could be capped off in a heparin or saline-lock as is in common use in hospitals.

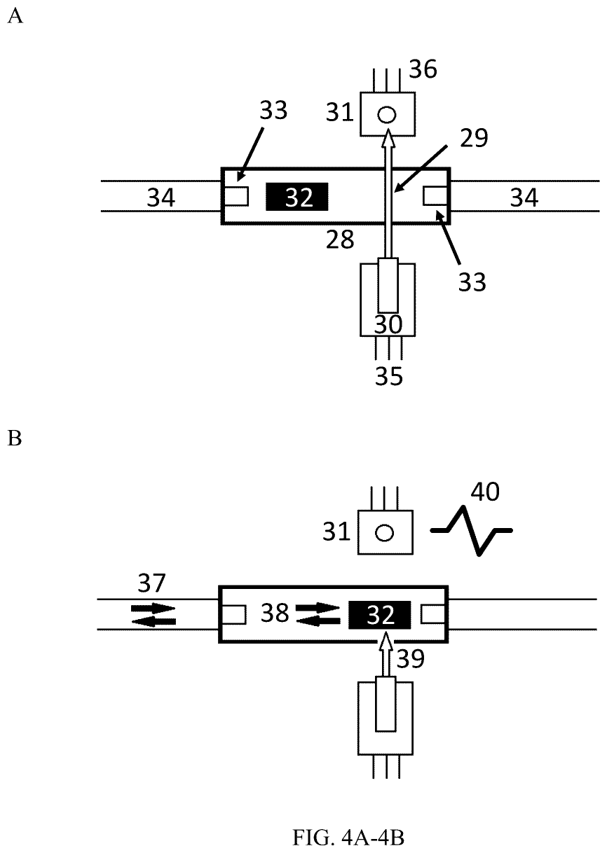

FIG. 4A-4B is a cross-section view of components 17-19 from FIG. 2A. In FIG. 4A, an optically engineered segment of IV tubing 28 allows light beams 29 to be passed readily through the tubing from a light source 30 to contact a light sensor 31. In this case a laser light source 30 was used to obtain experimental results but in other embodiments the light source could be a light emitting diode (LED) or other light source such as found commonly in smart watches and smart phones of any light frequency or multiple light frequencies including infrared or other non-visible wavelength light. In some embodiments an aperture and/or light beam focusing mechanism would be present to optimize the optical signal. A floating shutter (or indicator) composed of a cylindrical bead of light-absorbing material 32 was suspended in the IV fluid in the optical segment of IV tubing 28 so as to be free to move within the chamber of the optical segment of IV tubing swept along with IV fluid pulsatile movement. Two narrowings of the IV tubing diameter 33 confined the movement of the floating shutter 32 to prevent it from migrating upstream or downstream in the connected IV tubing 34; in some embodiments the floating shutter would additionally be attached to the wall of the IV tubing by some tethering structure which would prevent migration; in other embodiments the floating shutter would be replaced by a valve or flap which was attached to the wall of the IV tubing; in other embodiments the signal would not be optical but due to a strain gauge; in other embodiments the data received from the sensor would be analog instead of digital. The light source is powered from the PLC (not shown) by connecting wires 35. The light sensor is powered by and sends digital or analog signals to the PLC by connecting wires 36.

FIG. 4A represents the starting location of the floating shutter 32. In FIG. 4B, when a back and forth pulse is delivered to the fluid in the IV tubing, indicated by the arrows 37, a back and forth movement 38 of the floating shutter 32 is also produced. As the shutter moves in front of the light beam traversing the optical segment of IV tubing the shutter blocks the light beam 39. The interruption of the light beam is detected by the light sensor and sent as a digital or analog signal 40 to the PLC. After the forward fluid pulse movement has been completed the backwards fluid movement carries the floating shutter 32 back to its starting location as shown in FIG. 4A. Each repeated pulse results in a new light break-beam event which is identified with a time label by the PLC. In other embodiments, the PLC could be alternatively programmed to identify light restoration events or duration of light interruption or duration of light illumination.

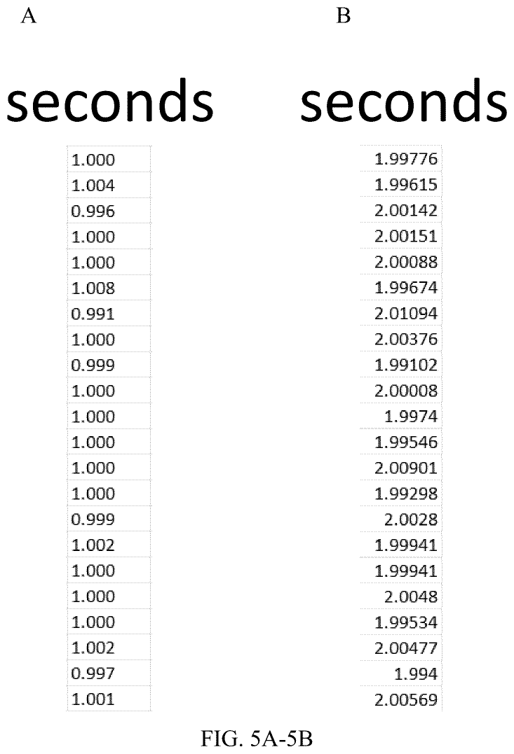

FIG. 5 shows experimentally derived values of inter-pulse intervals from the laser break-beam mechanism shown in FIG. 4. In FIG. 5A, a one second pulse pattern was programmed to be delivered by the pulse generator; the sample values in the column of FIG. 5A reflect the time calculated by the PLC between consecutive light break-beam events in seconds which demonstrated, for 60 recorded pulses, a mean inter-pulse interval of 1.000 sec with a standard deviation of 0.002 sec. In FIG. 5B, a two-second pulse pattern was programmed to be delivered by the pulse generator; the sample values in the column of FIG. 5B reflect the time calculated by the PLC between consecutive light break-beam events in seconds which demonstrated, for 60 recorded pulses, a mean inter-pulse interval of 2.000 sec with a standard deviation of 0.004 sec.

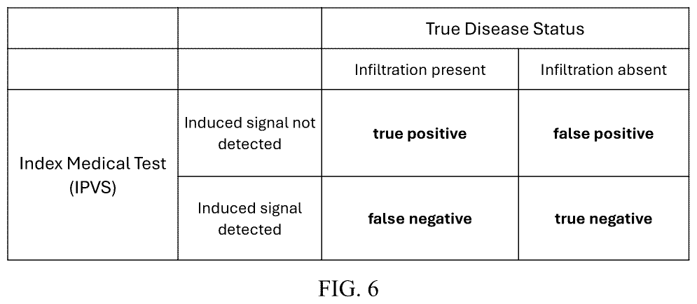

FIG. 6 shows a test performance measure table for IPVS functionality with respect to true disease status of PIV/vein infiltration present versus absent. In the case of a true positive test result, infiltration is present in the PIV and no light break-beam signal is detected with pulse introduction by the pulse generator. In the case of a true negative test result, no PIV infiltration is present and a light break-beam signal is detected with pulse introduction by the pulse generator. In the case of a false negative test result, infiltration is present in the PIV but a light break-beam signal is detected with pulse introduction by the pulse generator. In the case of a false positive test result, infiltration is not present in the PIV but no light break-beam signal is detected with pulse introduction by the pulse generator.

To optimize IPVS functionality, mechanisms for minimizing the likelihood of false positive and false negative test results are required.

In the case of false positives, the risk of mis-diagnosing a PIV as infiltrated or non-functional when in fact it was functional and patent would be that the PIV would unnecessarily be removed or not used when needed by nursing or other members of the care team out of concern for the development of or worsening of fluid or medication extravasation. This would entail time lost to the care team in that the time to place the mis-diagnosed PIV would have been lost, the time to remove the PIV would be lost, and the time to place a new PIV if it were needed would need to be expended. For the patient the risk would consist of a functional PIV not being available to be used if were needed acutely as well as the inconvenience and potential additional pain should a new PIV need to be placed. Additionally, for patients with a lack of good peripheral vein targets for PIV placement, due for example to extensive body surface area burns, the loss of a functional PIV may be catastrophic. Possible mechanistic causes of a false positive could consist of: failure of the pulse generator to successfully generate a pulse due to mechanical failure or inadequate contact with the IV tubing; failure of the floating shutter mechanism to reflect a patent, continuous fluid column due to the shutter mechanism being in some way disabled or stuck in place in the IV tubing or having dislodged or migrated; failure of the light source to generate a light beam; failure of the light sensor to detect the signal due to failure of the sensor; kinking of the IV tubing or the PIV itself that would prevent the induced pulse from being propagated; malfunction of the processing unit of the PLC; or other electrical system failures. Quality-control measures in manufacturing and clear product-insert instructions for use by care-team members would be central to minimizing these risks that largely revolve around device mechanical or electronic failure or improper end-user implementation.

In the case of false negatives, the risk of mis-diagnosing an infiltrated PIV as functional and patent would consist of the potential development and/or exacerbation of the very condition the IPVS system was meant to prevent, i.e. extravasation of IV fluids and/or medications into the soft tissues of a patient. This would entail pain and swelling for the patient at the extravasation site as well as potential infection and tissue damage. One possible mechanism of a false negative would be that the vein was infiltrated or thrombosed but the upstream IV tubing remained in a fluidic state that was able to conduct an induced pulse by the pulse generator that would be detected by the pulse detection sub-unit of the IPVS leading to the false conclusion that the downstream vein was patent. We have observed over many years of benchtop and in vivo experimentation that the risk of this scenario is minimized if not completely eliminated by incorporating a pulse dampener into the IV tubing 20 (FIG. 2A, reference (U.S. Pat. No. 11,666,700)); the path of least resistance of a propagated pulse in the IPVS system will always be downstream into the vein unless the vein is occluded, in which case the pulse will be absorbed by distention of the thin-walled dampener, removing the possibility of propagating a measurable pulse through the pulse detection sub-unit. Another possible mechanism of false negative would be that the vein was infiltrated or thrombosed but the IV tubing remained in a fluidic state that permitted movement of the floating shutter due to shaking or some other form of artifactual movement that would be detected by the pulse detection sub-unit of the IPVS and erroneously interpreted by an automated algorithm or a nurse or other care-provider as having arisen from an intended, induced pulse pattern. In the unpredictable and changing environment of a PIV in an awake patient in a clinic or hospital setting it is anticipated that artifactual movement could become an important factor in producing false negative test results ultimately leading to infiltration/extravasation events in a PIV that was erroneously deemed to be non-infiltrated. A mechanical restraint on the floating shutter such as a tether to the wall of the IV tubing or a flap mechanism attached to the wall of the IV tubing could be designed to constrain the movement of the floating shutter below a force threshold of the induced pulse thereby improving the odds that movement of the floating shutter would only occur in response to an induced pulse to prevent artifactual movement from generating a misinterpreted break-beam signal in the light sensor. Alternatively, a more secure mechanism for velocimetry detection based on strain-gauge or position sensors might replace shortcomings of optical technologies.

However, we believe that to inspire patient and clinician confidence in the validity of the IPVS system and to address in advance potential regulatory concerns, a more statistically grounded approach was necessary and with this motivation we sought to design an induced-pulse generation and analysis algorithm that would act as an informational lock-key mechanism to prevent false negative IPVS test results. This would be based on an induced pulse pattern that would be uncommon enough not to be likely to arise from random noise or patient movement and would be amenable to a simple, statistically significant method of confirming that the detected pulse matched the induced pulse within a defined confidence interval. In this way an observed break-beam signal originating from artifactual movement of the floating shutter could be filtered out as not similar enough to the expected induced pulse pattern by not attaining a defined statistical threshold, thereby eliminating the possibility of a false-negative reading. We sought to design this process as an automated algorithm to eliminate the potential error associated with the subjective interpretation of pulse patterns by human care-team members.

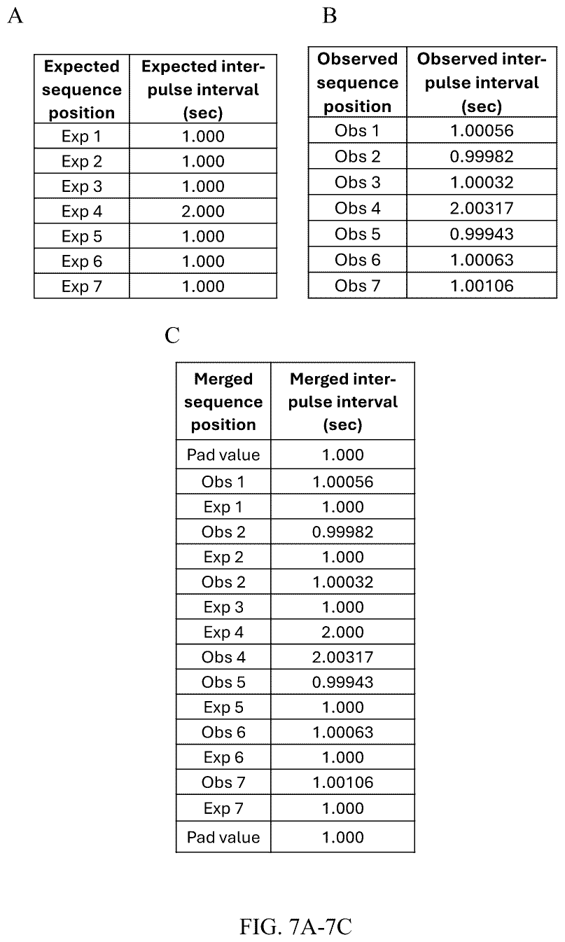

FIG. 7A shows a programmed pulse pattern labeled as “expected inter-pulse interval” delivered by the PLC to the pulse generating mechanism with inter-pulse intervals measured in seconds. This pulse pattern was programed to consist of a specific sequence of 7 inter-pulse intervals of either 1 second or 2 second duration. This schema provides for a total of 128 (2⁷) combinations of specific sequences of 1 or 2 second inter-pulse intervals whereby any given specific sequence of 7 values of 1 or 2 second inter-pulse intervals is 1/128 (0.8%) likely to arise randomly, i.e. it provides for greater than 99% certainty that a given sequence did not emerge from random noise. This is akin to a coin flip result being predicted correctly in 7 consecutive flips. FIG. 7B shows the sequence of the actual measured values of the inter-pulse intervals by the light break-beam sensor measured in seconds when the programmed pulse pattern was delivered into the fluid in the IV tubing, labeled as “observed inter-pulse interval”. This shows values very close to the expected inter-pulse interval values with some measured error due to standard deviation as shown experimentally in FIG. 5. The expected and observed inter-pulse interval sequence data tables were programmatically integrated by the PLC into a new data table, labeled as “merged inter-pulse interval”, as illustrated in FIG. 7C, through an inter-digitation of the expected-value and observed-value sequences. Padded values of 1.000 sec were appended as the first value and last value of this merged data table to bring the total number of values to 16 to put the data in a format to undergo Fast Fourier Transform (FFT), which requires the input data in the time domain to consist of 2N data points. In other embodiments some other number of observed and/or expected and/or padded values could be programmed into the algorithm to arrive at: greater certainty of a pulse sequence not arising from random noise at the cost of a greater duration in time to generate the data for the desired data set in the case of a greater number of pulses; or reduced time to achieve the desired data set in the case of fewer pulses at the expense of less certainty of a random result. In other embodiments only observed values would be analyzed without enmeshed expected values.

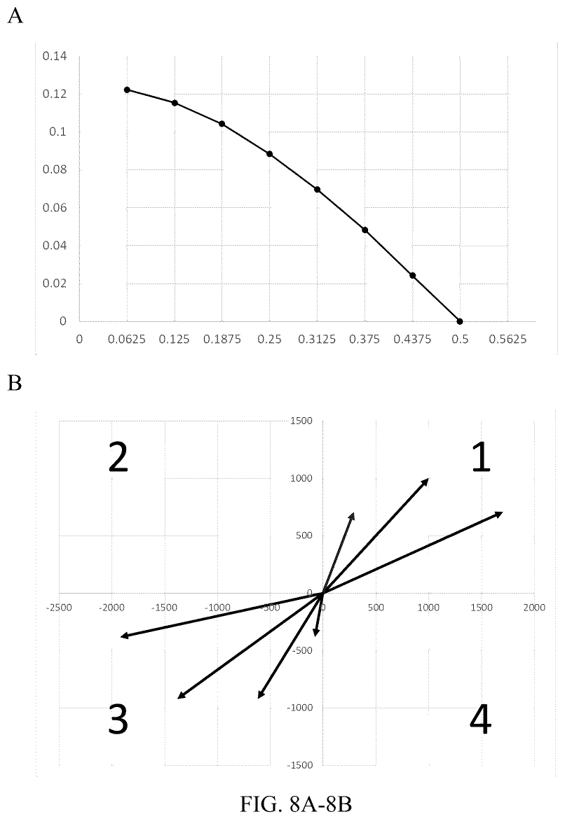

FIG. 8A shows a standardized form of a graph of an FFT frequency spectrum, as will be readily recognized by individuals knowledgeable in the art, generated from the data table shown in FIG. 7C, where frequency is shown on the x-axis and magnitude of the vector for a given frequency is shown on the y-axis. In this case, the FFT is used as a tool of functional data analysis (Ullah and Finch, BMC Med Res Methodol., 2013, 13:43) as it mathematically expands the pulse data from FIG. 7C into vector-field data with a unique and reproducible spectrum for any given pulse sequence which is more readily amenable to functional data analysis than the original time domain data. The FFT frequency spectrum observed in FIG. 8A is broad-band having a curve in the frequency domain that is readily amenable to regression analysis; this was performed by programmed algorithm by the PLC in real-time. In the spectral pattern shown in FIG. 8A vector magnitude varied by frequency according to a statistically significant quadratic association with coefficient of determination (R²) of 0.9994, p=0.00003, 99% confidence interval (−0.487, −0.274). No other 7-value sequence of 1 second and 2 second pulses or any other values for inter-pulse intervals can produce a spectral curve with significance at the 99% confidence interval for quadratic regression given the constraint of the input of the fixed values for expected inter-pulse interval and padded values into the time domain data. This allows only one possible sequence of generated pulses, i.e. the expected sequence programmed into the PLC, to result in measured, observed inter-pulse interval values that will produce the frequency spectrum shown in FIG. 8A. Any and all other observed pulse sequences will be filtered by the PLC as inadequately statistically congruent to the expected sequence and rejected as a signal match. The curve of the frequency spectrum (FIG. 8A) generated through this algorithm provides a means of rapidly assessing the similarity of two data sets with statistical confidence, in this case the two data sets being expected and observed pulse values of the IPVS system. Any deviation of the observed from the expected inter-pulse interval values will produce a greater or lesser distortion of the frequency spectrum curve which can be analyzed statistically in near real-time by programmatic definition of the limits of tolerance.

FIG. 8B shows the complex vector expression of the FFT curve from FIG. 8A. Vectors here are represented in conventional form with the x-axis representing the real number axis and the y-axis representing the imaginary axis. This shows that for the FFT generated from the data set of FIG. 7C, all vectors fall into the 1^{st} and 3^{rd} quadrants of the complex plane. Analysis of FFT vector distribution represents an additional data source for analysis or graphical expression.

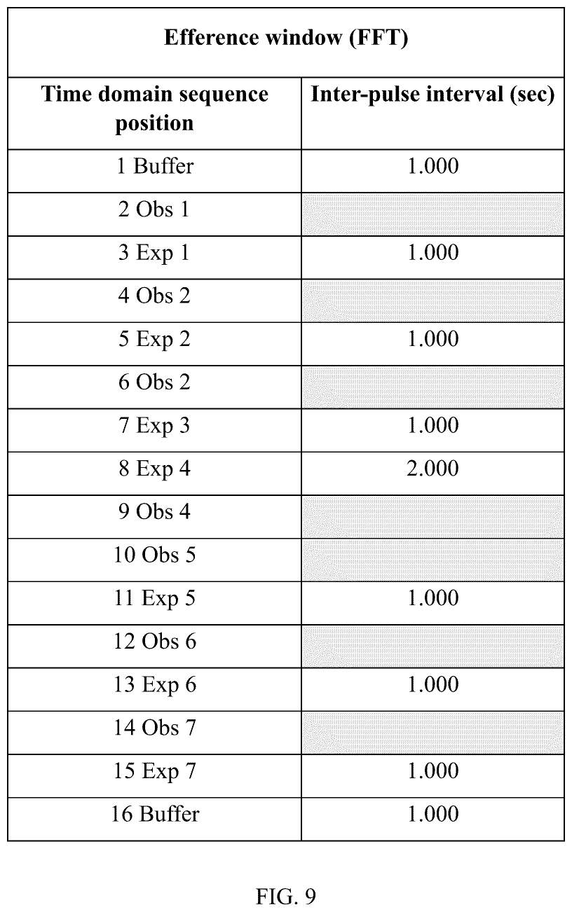

In FFT analysis, windowing of time-domain data is well known in the art (Oppenheim and Schafer, Discrete-time Signal Processing, Germany, Pearson, 2010). In the current application, the time domain data of 7 pulse-sequence data (FIG. 7B) is inserted algorithmically by the PLC into the gray shaded empty slots of an FFT “efference” window as illustrated in FIG. 9. This FFT window is asymmetric with respect to the placement of a single 2 second inter-pulse interval value to be able to select for an observed pulse sequence having a 2 second pulse in one and only one location within the input sequence. All other placements of the 2 second inter-pulse interval in the observed data will cause enough distortion in the FFT frequency spectral curve to be readily distinguished by analysis of the R² value of the curve in quadratic regression analysis, which in this case is being used as a quantitative assessment of goodness-of-fit of the frequency spectrum curve. In other embodiments linear or polynomial regression could replace quadratic regression as the test of goodness-of-fit of the spectral curve. In other embodiments vector distribution in the complex plane as in FIG. 8B could be used as data for analysis. In other embodiments, the FFT of the observed data could provide a tool for automated calibration of the pulse generating apparatus by rapid assessment of pulse delivery being too rapid or too slow and allowing the PLC to make automated adjustments in the speed of pulse generation based on distortions in the frequency spectrum curve.

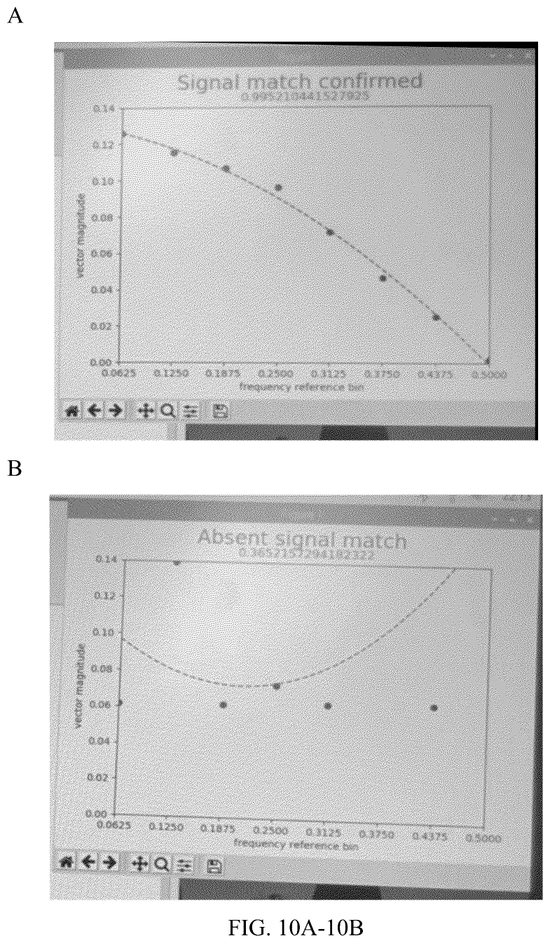

FIG. 10 shows graphs obtained in real time from a PLC (Raspberry Pi 4) programmed with the coding language, Python, demonstrating the functionality of the IPVS system. The pulse generator was programmed to deliver pulses to the IV tubing in the following sequence of expected inter-pulse intervals: 1 sec, 1 sec, 1 sec, 2 sec, 1 sec, 1 sec, 1 sec. The following observed inter-pulse interval values were measured by the laser sensor: 0.994 sec, 1.034 sec, 0.981 sec, 2.050 sec, 0.996 sec, 1.035 sec, 0.989 sec. These values, along with padded values, were inserted into the efference FFT window shown in FIG. 9 and the FFT was performed. The FFT spectrum along with quadratic regression line and R² value were graphed in real time. In the Python coding a cutoff value for R² of 0.98 and y-intercept of 0.1-0.14 were programmed as thresholds to confirm adequate statistical conformation of the observed to the expected signal. In FIG. 10A, those thresholds were met with R²=0.995 . . . and the PLC was programmed to print text in green, “Signal match confirmed”; by extension this allows for the conclusion that the PIV is not infiltrated. Alternatively, if no signal was received during a designated signal testing period, the PLC was programmed to print in red text, “Absent signal match”; by extension this allows for the conclusion that the PIV is infiltrated. To test the ability of the efference FFT technique to filter out artifactual pulse sequences, the following observed inter-pulse interval sequence was assayed: 2.040 sec, 0.996 sec, 1.034 sec, 0.980 sec, 2.046 sec, 0.996 sec, 1.038 sec. These observed values were all roughly equal to 1 sec or 2 sec inter-pulse intervals but were out of phase with the expected pattern of: 1 sec, 1 sec, 1 sec, 2 sec, see, 1 sec, 1 sec. After insertion into the efference FFT window from FIG. 9 and graphing of FFT spectrum, quadratic regression curve, and R² value, these observed values yielded the graph shown in FIG. 10B. Based on the cutoff value for R² of 0.98 and y-intercept of 0.1-0.14 the thresholds to confirm adequate statistical conformity of the observed to the expected signal were not met in this case with the calculated R² value of 0.365 . . . , and the PLC was therefore programmed to print in red text: “Absent signal match”; by extension this allows for the conclusion that a signal was present but artifactual and therefore the PIV is not safe to use; the PIV is either infiltrated or the IPVS system needs to undergo re-calibration and re-assessment.

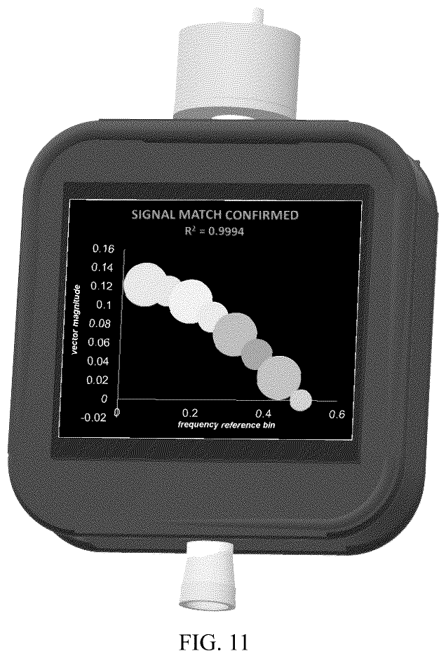

FIG. 11 shows one example of the system, the outer case of the IPVS system with the device monitor displaying a graphic interface in the form of a scatter plot/bubble chart as informed by the graphs in FIG. 10. In this case, in addition to the scatter plot curve display of vector magnitude on the y-axis and frequency on the x-axis, a bubble component is used to indicate phase data of the vectors through the size of the bubble with largest bubbles in the 1^{st} quadrant of the complex plane becoming progressively smaller until the 4^{th} quadrant of the complex plane and vectors falling on an axis having the smallest bubble. This functionality may be useful in certain embodiments in which 2 sec inter-pulse intervals are placed at different locations in the 7 pulse sequence than described here and the vectors fall into other quadrants of the complex plane or are tailored to fall into only one quadrant of the complex plane. For example, there may be a regulatory requirement to alter the pulse sequence intermittently. The delivered, or expected, inter-pulse interval sequence could be changed to 1 sec, 2 sec, 1 sec, 1 sec, 1 sec, 1 sec, 1 sec without altering the 99% certainty that the sequence was not due to random noise. In this case the efference FFT window would have to be shifted to accommodate the shifted position of the 2 sec inter-pulse interval. The frequency spectrum curve would be identical to the curve described in FIG. 8A but the vectors would reside in different quadrants of the complex plane from those in FIG. 8B leading to a different, but predictable, bubble chart pattern which could be used by the PLC to further confirm a pulse signal match.

This graphical and alphanumeric interface allows the practitioner and/or patient to see immediately that the signal being measured by the pulse detector is the same pulse that was generated by the pulse generator within a high degree of statistical confidence. This indicates that there is a very low likelihood of the observed pulse signal being artifactual and therefore a high confidence that the PIV is safe for use.

In certain aspects the pulsing within the IV catheter reduces the risk of infection. Catheter-related bloodstream infections (CRBSIs), occur when bacteria or other pathogens enter the bloodstream through an IV line, posing serious health risks. These infections can stem from prolonged catheter use, which allow microorganisms to colonize the catheter or surrounding skin. It is known that movement, when carefully managed in the context of intravenous (IV) therapy, plays a key role in reducing infection risk. Pulsing movement, such as intermittent or rhythmic motion in the context of intravenous (IV) therapy, can help reduce infection risk by disrupting conditions that promote bacterial growth. This type of movement, whether through patient mobility or mechanical aids like pulsatile flushing of IV lines, prevents the stagnation of fluids and blood at the catheter site, which can otherwise serve as a breeding ground for pathogens. Pulsing actions can also minimize biofilm formation—a protective layer created by bacteria on catheter surfaces—by dislodging early microbial attachments.

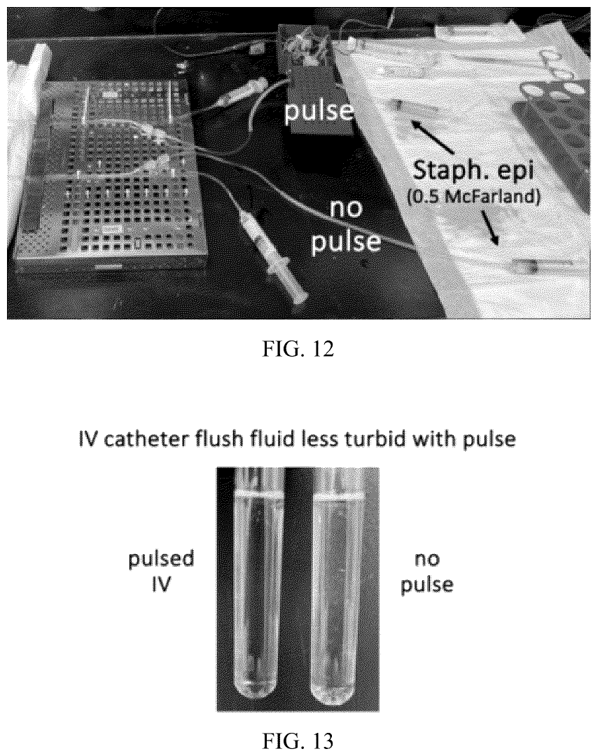

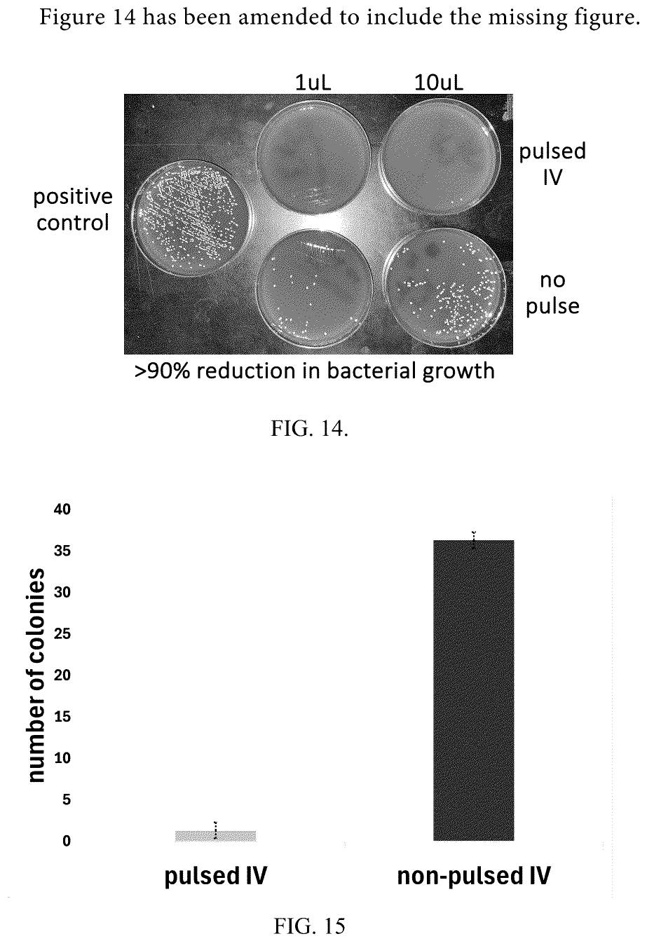

In certain embodiments the device can provide pulsation through the IV catheter. This pulsing movement reduces risk of infection. Studies have shown decreased bacterial growth. By creating a dual system allowing for pulsation of one IV catheter with a parallel control which does not receive pulsation the inventors have been able to isolate and quantify the bacterial growth in each condition (FIG. 12). FIGS. 13-15 show the pulsated IV catheter results in decreased bacterial growth. Ultimately this decreased growth will result in decreased IV infections for patients.

1. An intravenous fluid monitoring device comprising:
a body having a flow column, the flow column containing a flow indicator configured for axial fluid movement along the column;
a monitor window configured to allow detection of flow indicator movement;
a pulse generator, the flow indicator being in fluid communication with the pulse generator, the pulse generator configured to provide an efferent force to move the flow indicator in the flow column having a pulse frequency pattern, wherein after the efferent force stops an afferent force returns the flow indicator to an original position; and
an analysis module coupled to the monitoring device that is configured to monitor frequency spectra of induced pulse patterns, the analysis confirming catheter patency or lack thereof.

2. The device of claim 1, wherein the analysis module is comprised in a smart watch or smart phone.

3. The device of claim 1, wherein the analysis module is configured to produce a signal when patency is lost.

4. The device of claim 3, wherein the signal is a visual signal or an audible signal or a tactile signal, or combinations thereof.

5. A method for detecting intravenous infiltration comprising:
introducing a series of fluid pulses to a flow column in fluid communication with an intravenous catheter positioned within a subject to producing a pulse pattern within the flow column, the fluid pulse moving a flow indicator within the flow column along the long axis of the flow column;
monitoring the movement of the flow indicator across an observation location to obtain the pulse pattern, wherein movement of the flow indicator across the observation location is detected;
analyzing the movement of the flow indicator by converting a data signal, analyzing the data signal using a programmable logic circuit (PLC) and conducting algorithmic analysis of the data signal determining if the data signal is congruent with an expected pulse pattern within a statistical confidence interval.

6. The method of claim 5, wherein the data signal is processed by Fast Fourier Transform (FFT).

7. The method of claim 5, further comprising displaying a graphical or alpha numeric representation of the analysis on a user interface.

8. The method of claim 7, wherein the user interface is a smart watch or smart phone.

9. A system for performing frequency analysis of pulse patterns in a fluid column comprising:
a pulse generator operatively coupled to the fluid column;
a pulse detection module operatively coupled to the fluid column;
a frequency analysis module operatively coupled to the detection module, the analysis module configured to receive a series of data points forming a signal from the detection module and verify that the signal detected in the fluid column is congruent to an induced pulse pattern to verifying that the fluid column is fluidically patent and continuous.

10. The system of claim 9, wherein the frequency analysis module is configured to perform Fast Fourier Transform (FFT) on data generated by the detection module.

11. The system of claim 10, wherein a frequency spectrum of the FFT is a broad-band spectrum analyzed by linear, quadratic, or polynomial regression deriving a goodness-of-fit model based on a statistical confidence interval.

12. The system of claim 11, wherein the phase data of the FFT can be analyzed to contribute to a statistical confidence interval.

13. The system of claim 9, wherein the induced pulse patterns consist of small data sets of inter-pulse interval patterns which vary according to a binary number system.

14. The system of claim 9, wherein efference FFT window is used to enmesh an expected inter-pulse interval data pattern with an observed inter-pulse interval data pattern to arrive at an enmeshed data set and obtain the frequency spectrum of the enmeshed data set.

15. The system of claim 9, wherein expected, observed, and enmeshed data sets consist of small data sets of inter-pulse interval values which vary according to a binary number system.

16. The system of claim 14 or 15, wherein aberrations in the frequency spectrum can be analyzed for autocalibration of the pulse generating system in cases where pulses are being delivered too quickly or too slowly.

17. The system of claim 9, wherein pulse data is obtained by an optical break-beam method.

18. The system of claim 9, wherein pulse data is obtained by a strain gauge method employing pulse data in digital or analog form.

19. The system of claim 9, wherein pulse detection occurs upstream from a point in the fluid column that may be interrupted with respect to a continuous fluid channel.

20. The system of claim 9, wherein the fluid column is intravenous tubing attached to a peripheral intravenous catheter in a patient vein.

21. The system of claim 9, wherein in which the fluid column is a vein.

22. The system of claim 9, wherein in which the fluid column is an artery.

23. The system of any one of claims 9 to 22 in which a programmable logic circuit performs all steps in an automated manner including: generating the unique pulse pattern, detecting the pulse pattern, performing FFT and statistical analysis, printing out results on a display monitor to be viewed by patients or members of a medical care team.

24. The system of any one of claims 9 to 23 in which the FFT frequency spectrum and/or FFT phase data are displayed on a smart watch or smart phone display in scatter plot, bubble graph, or other graphical format.
